(19) United States
(12) Patent Application Publication
(10) Pub. No.: US 2016/0008522 A1
(43) Pub. Date: Jan. 14, 2016

(54) DRUG COATING LAYER, METHOD OF CONTROLLING MORPHOLOGICAL FORM OF DRUG COATING LAYER, MEDICAL DEVICE, AND METHOD OF DELIVERING DRUG

(71) Applicant: TERUMO KABUSHIKI KAISHA, Tokyo (JP)

(72) Inventors: Keiko YAMASHITA, Tokyo (JP); Hiroshi GOTOU, Kanagawa (JP); Youko TERAJIMA, Kanagawa (JP); Eisuke FURUICHI, Kanagawa (JP)

(73) Assignee: TERUMO KABUSHIKI KAISHA, Tokyo (JP)

(21) Appl. No.: 14/860,137

(22) Filed: Sep. 21, 2015

Related U.S. Application Data
(63) Continuation of application No. PCT/JP2014/059668, filed on Apr. 1, 2014.

(30) Foreign Application Priority Data
Apr. 1, 2013 (JP) 2013-076046

(51) Int. Cl. A61L 31/16; A61L 31/08

(52) U.S. Cl. CPC A61L 31/16; A61L 31/08; A61L 2300/606; A61L 2420/02; A61L 2300/416

(57) ABSTRACT

A drug coating layer which ensures that when a medical device coated with a drug is delivered into a living body, the drug can be prevented from peeling off during delivery operation through a body lumen or cavity such as a blood vessel; and/or a drug coating layer excellent in transferability of a drug to a target tissue; and a method of controlling the morphological form of a drug coating layer are provided. The drug coating layer, which is formed on a substrate surface and contains a water-insoluble drug, has at least one morphological form selected from the group consisting of defined morphological forms (1) to (4)

### CROSS-REFERENCES TO RELATED APPLICATIONS

This application is a continuation of International Application No. PCT/JP2014/059668 filed on Apr. 1, 2014, and claims priority to Japanese Application No. 2013-076046 filed on Apr. 1, 2013 the entire content of both of which is incorporated herein by reference.

### TECHNICAL FIELD

The present disclosure relates to a drug coating layer for a drug eluting medical device, a method of controlling the morphological form of a drug coating layer, a medical device, and a method of delivering a drug.

### BACKGROUND DISCUSSION

In recent years, development of drug eluting balloons (hereinafter sometimes referred to as DEB) wherein a balloon catheter is coated with a drug has been made, and it has been reported that such drug eluting balloons are effective in treating and preventing restenosis. The balloon is coated with a coating layer which contains a drug and excipients, such that when a blood vessel is dilated, the balloon is pressed against the blood vessel wall and the drug is delivered to a target tissue.

JP-T-H 09-500561 describes a DEB, which includes a balloon (expandable portion) formed of a polymer. The balloon is structured to have a coating layer in which a biologically active agent is contained in a releasable manner.

It is an object of the present disclosure to provide a drug coating layer capable of preventing a drug from peeling off during the delivery operation into and through a body cavity such as a blood vessel, in a process wherein a medical device is coated with a drug and delivered into a living body, and/or a drug coating layer excellent in transferability of a drug to a target tissue, a method for controlling the morphological form of a drug coating layer, and a medical device using the drug coating layer and the controlling method.

### SUMMARY

The present disclosure has been made on the basis of a finding that drug coating layers having a specified morphological form (hereinafter sometimes referred to as form) are excellent drug coating layers. A medical device provided with a drug coating layer on a surface thereof according to the present disclosure has at least one characteristic selected from the following:

1) being a drug eluting medical device such that a drug is insusceptible to peeling-off during delivery process to a target tissue; and

2) being excellent in transferability of a drug to a target tissue.

Specifically, the present disclosure provides the following aspects:

[1] A drug coating layer formed on a substrate surface, wherein a water-insoluble drug coating layer has at least one morphological form selected from the group consisting of the following morphological forms (1) to (4): (1) a first morphological form in which a substantially plate-shaped amorphous phase is predominant at a surface and inside the whole drug coating layer; (2) a second morphological form in which small incomplete crystals are predominant; (3) a third morphological form in which small incomplete crystals in a network form are present in at least a portion of the drug coating layer; and (4) a fourth morphological form in which needle- or rod-shaped or spheroidal crystals are present in at least a portion of the drug coating layer.

[2] The drug coating layer as described in [1], wherein the drug coating layer has at least one morphological form (hereinafter, morphological form will be referred to as form) selected from the group consisting of the first form and the second form, namely, (1) the first form in which a substantially plate-shaped amorphous phase is predominant at a surface and inside the whole drug coating layer, and (2) the second form in which small incomplete crystals are predominant, and the first form and the second form contain a same drug or different drugs, and formation of the first form and the second form can be controlled by changing conditions for formation of the drug coating layer.

[3] A method of controlling the morphological form of the drug coating layer as described in [1] or [2], wherein at the time of forming the drug coating layer by applying onto a substrate a drug coating composition which contains a water-insoluble drug and a glycerin-free solvent, the rate of removal of the solvent is regulated. Note that “by applying” means, here and hereafter, application in a broad sense to be described later.

[4] The method of controlling the morphological form of the drug coating layer as described in [3], wherein the solvent is tetrahydrofuran, and ethanol.

[5] The method of controlling the morphological form of the drug coating layer as described in [3], wherein the water-insoluble drug is rapamycin, paclitaxel, docetaxel, or everolimus.

[6] The method of controlling the morphological form of the drug coating layer as described in [3], wherein the coating composition does not contain water.

[7] A medical device which is provided on a surface thereof with the drug coating layer as described in [1] or [2], is delivered in a radially contracted state when delivered in a living body, and is radially expanded locally so as to release a drug from the drug coating layer.

[8] A method of delivering a drug, including: delivering the medical device as described in [7] into a lumen, radially expanding an expandable portion possessed by the medical device, and allowing a drug coating layer provided on the expandable portion to act on the lumen.

### BRIEF DESCRIPTION OF THE DRAWINGS

FIG. 1 is a drawing which shows a scanning electron microscope (hereinafter sometimes referred to as SEM) image (2000 fold) of a drug coating layer of [Example 1];

FIG. 2 is a drawing which shows an SEM image (1000 fold) of a drug coating layer of [Example 4];

FIG. 3 is a drawing which shows an SEM image (2000 fold) of a drug coating layer of [Example 5];

FIG. 4 is a drawing which shows an SEM image (2000 fold) of a drug coating layer of [Example 7];

FIG. 5A is a drawing which shows an SEM image (2000 fold) of a drug coating layer of [Example 8A], and FIG. 5B is a drawing which shows an SEM image (1000 fold) of a drug coating layer of [Example 8B], FIG. 5A and FIG. 5B being drawings which each show SEM images of the drug coating layer of [Example 8] of the same drug eluting balloon obtained from a coating solution 8, taken at different points of the surface of the drug eluting balloon;

FIG. 6A is a drawing which shows an SEM image (2000 fold) of a drug coating layer of IN.PACT of a comparative example [Example 10A], and FIG. 6B is a drawing which shows an SEM image (2000 fold) of a drug coating layer of IN.PACT of a comparative example [Example 10B], FIG. 6A and FIG. 6B each showing SEM images of different portions of [Example 10];

FIG. 7 is a graph showing the results of Evaluation 1 and Evaluation 2, wherein an outlined bar represents the remaining rate (mass %) of PTX after wrapping and a filled bar represents the remaining rate (mass %) of PTX after (wrapping+delivery), and Nos. are No. 4 (Example 4), No. 5 (Example 5), No. 7 (Example 7), and No. 8 (Example 8); and

FIG. 8 is a sectional schematic view of an experimental system in a state wherein a balloon catheter 3 with a balloon 4 is inserted in a guiding catheter 2 disposed in a mimic blood vessel 1, in a drug coating layer durability evaluation test using the mimic blood vessel 1 in Evaluation 2.

### DETAILED DESCRIPTION

A drug coating layer formed on a surface of a substrate,

wherein the coating layer of a water-insoluble drug has at least one morphological form selected from the group consisting of the morphological forms (1) to (4) described below.

Here, the group of forms are:

(1) a first form in which a substantially plate-shaped amorphous phase is predominant at a surface and in an inside of the whole of the drug coating layer;

(2) a second form in which small incomplete crystals being about to be crystals are predominant;

(3) a third form in which small incomplete crystals in a network form are present in at least a portion thereof; and

(4) a fourth form in which needle- or rod-shaped or spheroidal crystals are present in at least a portion thereof.

The water-insoluble drug may be the same drug or different drugs in the first to fourth forms.

Preferably, the following first and second drug coating layers are provided.

The first drug coating layer is a drug coating layer such that the drug coating layer has at least one form selected from the group consisting of the following first and second forms, namely,

(1) the first form in which a substantially plate-shaped amorphous phase is predominant at a surface and inside the whole drug coating layer, and

(2) the second form in which small incomplete crystals are predominant, and

wherein the first form and the second form contain a same drug or different drugs, and formation of the first form and the second form can be controlled by changing conditions for formation of the drug coating layer.

(1) First form (A): Substantially plate-shaped amorphous phase is predominant.

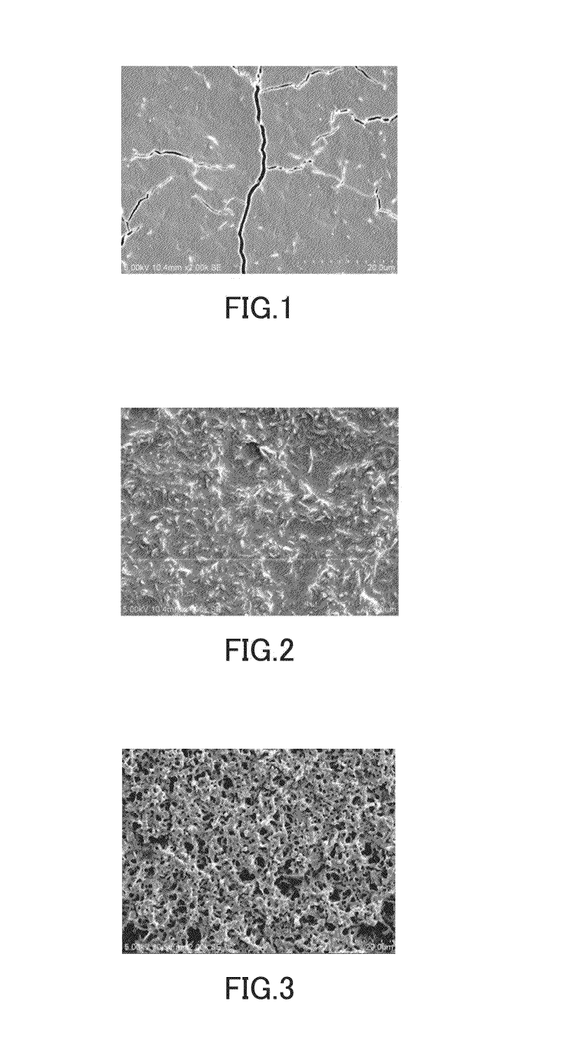

The first form (A) is a drug coating layer of form (A) wherein an amorphous phase is predominant. As an example of this, there may be mentioned a surface or the like of a drug coating layer in [Example 1] obtained by applying a first drug coating composition to a balloon surface. The surface of [Example 1] is represented by the SEM image in FIG. 1, and will be represented by 1(A) in Table 1 set forth later. A substantially plate-shaped amorphous phase is predominant at the surface and inside the whole drug coating layer. This form has a plate-shaped structure, has a flat and homogeneous surface, and has a continuous drug coating layer.

The term “predominant” herein means a form wherein not less than 50% of the area of a single visual field of an SEM image is occupied by the relevant region, and it is sufficient that the relevant form exists either at the surface or inside of the drug coating layer observed from a single balloon.

(2) Second form (B): Small incomplete crystals are predominant.

The second form (B) is a drug coating layer of a form (B) wherein small incomplete crystals, which are about to be crystals, are predominant, although amorphous phases are also present. This form means that the form in which a plurality of small incomplete crystals (which are about to be crystals) are observed to occupy not less than 50% of the area of a single visual field of an SEM image. As an example of this form, there may be mentioned a surface or the like (shown in FIG. 2) of a drug coating layer [Example 4] obtained by coating a balloon surface with a first drug coating composition which is a coating solution 4 to be described later. If such a form is present either at the surface or the inside of a drug coating layer observed from a single balloon, the drug coating layer is of the second form (B) wherein small incomplete crystals (which are about to be crystals) are predominant. In this form, the surface is not flat but has ruggedness (projections and recesses).

The surface of [Example 4] is shown in FIG. 2, is represented as 4(B) in Table 1 later, and has small incomplete crystals (which are about to be crystals) present predominantly therein.

The second drug coating layer is a drug coating layer such that the drug coating layer has at least one form selected from the group consisting of a third form and a fourth form, namely,

(3) a third form wherein small incomplete crystals in a network form are present in at least a portion thereof; and

(4) a fourth form wherein needle- or rod-shaped or spheroidal crystals are present in at least a portion thereof,

the third form and the fourth form containing the same drug or different drugs, the advent of the third form and the fourth form being controllable by changing conditions for formation of the drug coating layer.

(3) Third form (C): Small incomplete crystals in a network form are present in at least a portion of the drug coating layer.

The third form (C) is a form (C) wherein small incomplete crystals in a network form are present in at least a portion of the drug coating layer. As an example of the form at the surface or the inside of the drug coating layer, there may be mentioned a drug coating layer [Example 5] (shown in FIG. 3) or the like obtained by coating a balloon surface with a second drug coating composition which is a coating liquid 5 to be described later.

The embodiment where small incomplete crystals in a network form are predominant is a shape wherein as in [Example 5], individual crystals are not present independently, it is difficult to determine the size of a single crystal, and crystals are joined to each other in a network form. Note that [Example 5] is an SEM image taken by SEM, will be represented as 5(C) in Table 1 later, and has incomplete small crystals in a network form being present predominantly therein.

(4) Fourth form (D) is a form of a drug coating layer in the embodiment (D) where a crystalline form is predominant. The form of the surface or the inside may be exemplified by a surface or the like where crystals (spheroidal) are predominant, in drug coating layers [Example 7] and [Example 8] obtained by coating a balloon surface with second drug coating compositions which are coating solutions 7 and 8 described later. As for the crystalline shape, a single one or a mixed state of various shapes such as needle-like, rod-like and spheroidal shapes is observed.

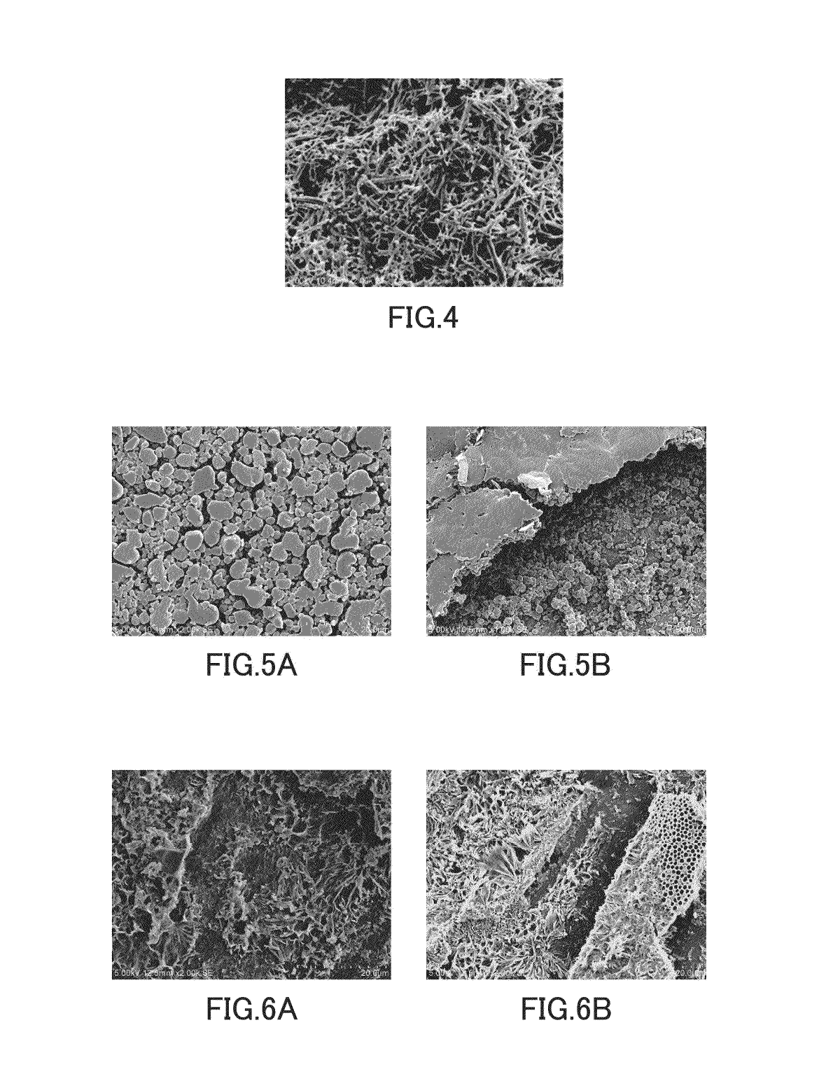

D1) An SEM image of a drug coating layer [Example 7] in the embodiment where crystals (needle- or rod-shaped) are predominant is shown in FIG. 4. The surface of [Example 7] is represented as 7(D1) in Table 1 later, and has crystals (needle- or rod-shaped) present predominantly therein.

D2) An SEM image of a drug coating layer in Example [Example 8] in the embodiment where crystals (spheroidal) are predominant or a surface thereof is covered with an amorphous film whereas spheroidal crystals are present in the inside thereof, is shown in FIG. 5A and FIG. 5B. FIG. 5A is represented as 8(D2) in Table 1 later, and has crystals (spheroidal) present predominantly therein. FIG. 5B is represented as 8(D3) in Table 1 later, and has a surface thereof covered with an amorphous film, with spheroidal crystals being present in the inside thereof. The spheroidal crystals obtained in the present disclosure have a surface which is not three-dimensional but is flat as if crushed down.

Each form of the group consisting of the forms (1) to (4) of the drug coating layer as above-mentioned can be obtained by a method in which at the time of coating a substrate with a drug coating composition containing a water-insoluble drug and a solvent to form a drug coating layer, the rate of removal of the solvent is regulated, to thereby control the form of the resulting drug coating layer.

Further, in forming the drug coating layer, a basic compound which is positively charged in a physiological pH range can be present in the drug coating layer forming site. Preferably, in the group of forms (1) to (4) of the drug coating layer, the basic compound positively charged in a physiological pH range is an amidine derivative represented by the following general formula (1-a) or (1-b). Each form of the drug coating layer can be controlled by use of a drug coating composition in which the amidine derivative and the water-insoluble drug are present in the state of being dissolved in the solvent.

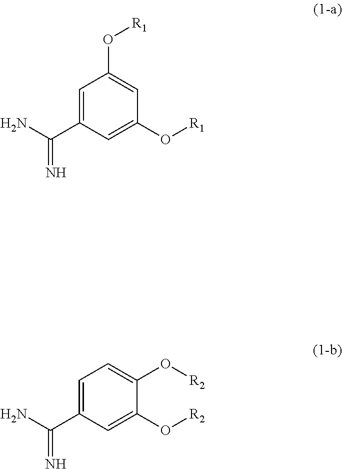

In the formulas, R₁ and R₂ are each independently an alkyl group of 10 to 18 carbon atoms.

The drug coating layer in the present disclosure is preferably obtained by the following method of controlling the forms using the following drug coating composition.

(1) First drug coating layer: For example, a coating composition obtained using a solvent not including glycerin is applied onto a substrate and dried, while the ratio of solvents is controlled to control the resulting form. By this control, it is possible to control a first drug coating layer thus obtained by evaporating away the solvent so that when a surface of the first drug coating layer is observed under a scanning electron microscope (SEM), the first drug coating layer contains at least one form selected from the group consisting of a first form wherein a plate-shaped amorphous phase is predominant at the surface or in the inside of the whole of coating layer and a second form wherein small incomplete crystals are recognized.

(2) Second drug coating layer: For example, a composition obtained using a solvent including glycerin is applied onto a substrate and dried, while the ratio of solvents is controlled to control the resulting form. By this control, it is possible to achieve such a control that a second drug coating layer thus obtained by evaporating away the solvent includes at least one form selected from the group consisting of a third form wherein small incomplete crystals in a network form are predominant and a fourth form wherein crystals having needle- or rod-shaped or spheroidal crystals are predominant. The embodiment where a crystalline form of the fourth form is present may include an embodiment where the surface is covered with an amorphous film and crystals are recognized to be present under the film (in the inside of the drug coating film).

The method of controlling the form of a drug coating layer of the present disclosure has the following characteristics.

(1) Water is not used in the solvent. When water is used in the present disclosure, water can be very little mixed into the solvent, so that the use of water in the solvent may cause the water-insoluble drug to separate out from the drug coating composition which is a precursor solution for the drug coating layer. As a result, the drug coating composition is instable.

(2) By controlling the ingredients of the solvent and the ratio of the ingredients, it is possible to control the form of the drug coating layer at normal temperature, without any special heat treatment (annealing).

(3) The form of the resulting drug coating layer can be easily controlled according to the presence/absence of glycerin, which is a lubricant, in the solvent.

(4) A drug coating layer having a specified form in a wide range, instead of having only a plurality of forms mixedly present therein, can be obtained by controlling the ingredients of the solvent and their ratio to within specific ranges.

Ingredients of the drug coating composition, which is a coating liquid for controlling the form of the first drug coating layer and the second drug coating layer, will be described below.

### [Water-Insoluble Drug]

The water-insoluble drug means a drug which is insoluble or difficultly soluble in water, specifically a drug whose solubility in water is less than 5 mg/mL at pH 5 to 8. The solubility may be less than 1 mg/mL, or even less than 0.1 mg/mL. The water-insoluble drug includes fatty-soluble drugs.

Some preferred exemplary water-insoluble drugs include immunosuppressants, for example, immunologically active agents such as cyclosporines (including cyclosporine), rapamycin, etc., carcinostatic agents such as paclitaxel, etc., antiviral or antibacterial agents, antineoplastic agents, analgesic and anti-inflammatory agents, antibiotic, antiepileptic, anxiolytic agents, antiparalytic, antagonists, neuron blocking agents, anticholinergic and cholinergic agents, muscarine antagonists and muscarine agents, antiadrenergic agents, antiarrhythmic agents, antihypertensive agents, hormone preparations, and nutritional supplements.

The water-insoluble drug is preferably at least one selected from the group consisting of rapamycin, paclitaxel, docetaxel and everolimus. Rapamycin, paclitaxel, docetaxel and everolimus include their analogs and/or their derivatives so long as the analogs and/or derivatives have the same or equivalent efficacy to the original. For instance, paclitaxel and docetaxel are in the relation of analogs, whereas rapamycin and everolimus are in the relation of derivatives. Among these, more preferred is paclitaxel.

[Basic Compound which is Positively Charged in Physiological pH Range]

The content of the basic compound which is positively charged in a physiological pH range is not particularly limited. The content is preferably 0.5 to 100 parts by mass, more preferably 2 to 80 parts by mass, and further preferably 3 to 50 parts by mass, based on 100 parts by mass of the water-insoluble drug.

The physiological pH is the range of pH in a living body, mainly in blood. More specifically, the physiological pH is preferably pH 6.0 to 8.0. The basic compound which is positively charged in a physiological pH range is preferably a basic compound having an amidino group, a basic compound having two or more amino groups, a basic compound having a piperidine ring, or a basic compound having a quaternary amine, and is more preferably a compound represented by the following Formula 1.

The basic compound positively charged in a physiological pH range has an alkyl group-containing moiety, the hydrophobic property of which can enhance affinity for the water-insoluble drug and for a medical device surface, whereby peeling-off of the drug coating layer during the delivery process to a target tissue can be prevented. On the other hand, the moiety positively charged due to the amino groups can enhance affinity for cell surfaces which are negatively charged. Therefore, it is made possible to deliver a balloon catheter coated with the drug to a target tissue, without peeling-off of the drug during delivery through a body cavity, to press the balloon against a blood vessel wall simultaneously with expansion of the balloon, to rapidly release the drug, and to transfer the drug to the tissue.

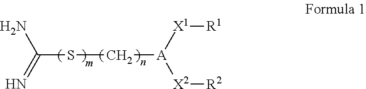

In the Formula 1, A represents an aromatic ring, R¹ and R², which may be the same or different, each represent an alkyl or alkenyl group of 10 to 25 carbon atoms; X¹ and X², which may be the same or different, each represent —O—, —S—, —COO—, —OCO—, —CONN— or —NHCO—; m is 0 or 1, and n is 0 or an integer of 1 to 6.

Preferably, the basic compound is a compound wherein m=0 and n=0 so that the moieties are linked directly, and is preferably the following amidine derivative.

### [Amidine Derivative]

An amidine derivative represented by the following general formula (1-a) or (1-b) is preferable.

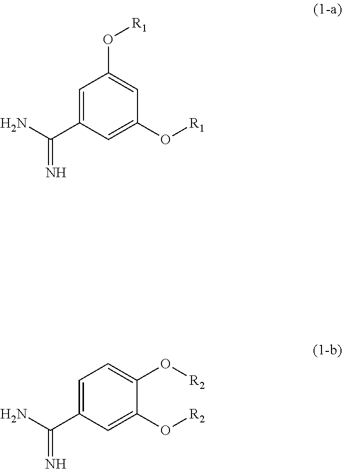

In the formulas, R₁ and R₂ are each independently an alkyl group of 10 to 18 carbon atoms.

A method of synthesizing these amidine derivatives is described in Japanese Patent No. 3919227, the content of which is incorporated by reference. Preferable amidine derivatives are exemplified below.

3,5-Dipentadecyloxybenzamidine represented by the following formula (3) (hereinafter sometimes referred to as TRX-20)

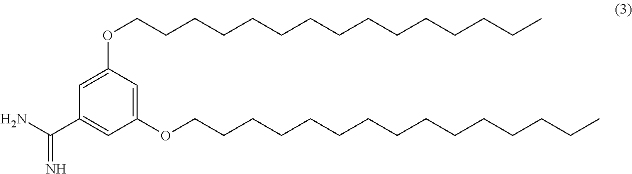 [SMILES: CCCCCCCCCCCCCCCOc1cc(OCCCCCCCCCCCCCCC)cc(C(=N)N)c1]

3,5-Dihexadecyloxybenzamidine represented by the following formula (4)

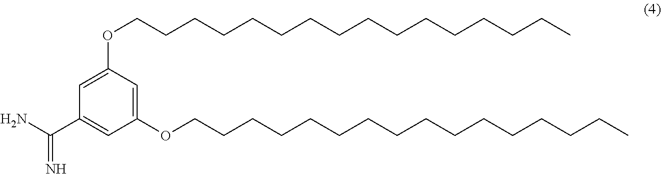 [SMILES: CCCCCCCCCCCCCCCCOc1cc(OCCCCCCCCCCCCCCCC)cc(C(=N)N)c1]

3,5-Dioctadecyloxybenzamidine represented by the following formula (5)

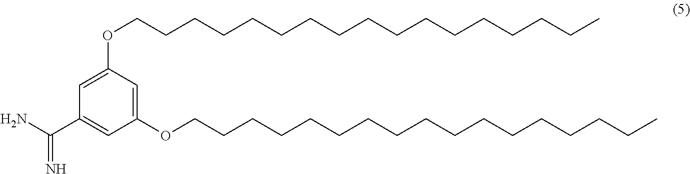 [SMILES: CCCCCCCCCCCCCCCCCOc1cc(OCCCCCCCCCCCCCCCCC)cc(C(=N)N)c1]

3,5-Didecyloxybenzamidine represented by the following formula (6)

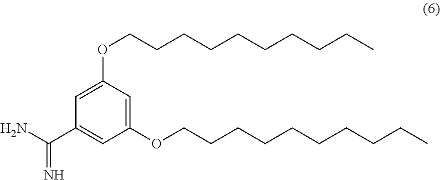 [SMILES: CCCCCCCCCCOc1cc(OCCCCCCCCCC)cc(C(=N)N)c1]

3,4-Didecyloxybenzamidine represented by the following formula (7)

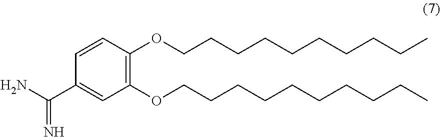 [SMILES: CCCCCCCCCCOc1ccc(C(=N)N)cc1OCCCCCCCCCC]

The basic compound positively charged in a physiological pH range has the hydrophobic moiety, whereby affinity for the water-insoluble drug and for a medical device surface can be enhanced. On the other hand, the moiety positively charged due to the amino groups can enhance affinity for cell surfaces, whereby the drug linked to the hydrophobic moiety can be efficiently transferred to the cell structure.

### [Solvent]

The solvent for applying the above-described water-insoluble drug and the basic compound positively charged in a physiological pH range onto a substrate is not specifically restricted, so long as the solvent can permits these ingredients to be dissolved or dispersed therein. Examples of the solvent include tetrahydrofuran, ethanol, acetone, methanol, dichloromethane, hexane, ethyl acetate, and water. Specific explanations of these will be given in the following descriptions of the drug coating layers. It is preferable not to use water in the solvent. A water-containing solvent would become instable, thereby causing the water-insoluble drug to separate out in a precursor solution prior to forming the drug coating layer.

Though not restricted, the drug coating layer can be formed by application of an immersion method (dipping method), an applying method, a pipetting method, a spraying method, a spin coating method, a mixed solution-impregnated sponge coating method or the like. Among these methods, preferred is the pipetting method. Preferably, with a balloon kept expanded, a drug coating layer is formed thereon. Herein, these methods may be generically described as “applying” in its broad sense.

In the following, the points in which the first and second drug coating compositions differ from each other will be mainly described. The points which will not be described below are common to the first and second drug coating compositions and common to the first and second drug coating layers.

### (1) First Drug Coating Composition and First Drug Coating Layer

1) The first drug coating composition is a composition wherein the water-insoluble drug and the amidine derivative are present in the state of being dissolved in a solvent which does not contain glycerin.

### [Solvent]

Indispensable solvents: tetrahydrofuran (hereinafter sometimes described as THF) and ethanol (hereinafter sometimes described as EtOH). The tetrahydrofuran is used as a solvent for dissolving the amidine derivative. Other solvents than the above-mentioned which can be added to the first drug coating composition include acetone, methanol, dichloromethane, hexane, and ethyl acetate.

### 2) Preparation of the First Drug Coating Composition

The amidine derivative is dissolved in tetrahydrofuran.

The water-insoluble drug is dissolved in ethanol, acetone, tetrahydrofuran, or a mixed solvent thereof.

Both the above-mentioned solutions are prepared and then mixed with each other, to obtain the first drug coating composition.

### 3) First Drug Coating Layer

It is preferable to form the first drug coating layer on a substrate surface of a medical device by use of the first drug coating composition.

The relation between the solvent in the first drug coating composition and the drug coating layer:

By controlling the ratio between the solvents, the first form (A) wherein a substantially plate-shaped amorphous phase is predominant at the surface and in the inside of the first drug coating layer and the second form (B) wherein small incomplete crystals being about to be crystals are recognized are obtained. Tetrahydrofuran is a good solvent for both the amidine derivative and the water-insoluble drug, and is high in volatility. Therefore, as the proportion of the tetrahydrofuran is higher, the amorphous phase is more predominant in the first drug coating layer.

The drug coating layer [first form (A)] has the substantially plate-shaped amorphous phase predominant at the surface and in the inside of the whole of the drug coating layer. A preferred solvent ratio (mass %) is as follows.
TFH EtOH, Acetone, or EtOH/Acetone mixed liquid
Not less than 65%, Not less than 0%,
not more than 100% not more than 35%

The drug coating layer [second form (B)] has small incomplete crystals (which are about to be crystals) predominant therein. A preferred solvent ratio (mass %) is as follows.
TFH EtOH, Acetone, or EtOH/Acetone mixed liquid
Not less than 1%, Not less than 85%,
not more than 15% not more than 99%

[First Form (A): SEM Photograph of Drug Coating Layer in the Embodiment where a Substantially Plate-Shaped Amorphous Phase is Predominant]

The form of the surface or the inside of a drug coating layer of form (A) wherein an amorphous phase is predominant can be exemplified by surfaces or the like of drug coating layers [Example 1], [Example 2], and [Example 3] which are obtained by coating a balloon surface with the first drug coating composition which is coating solutions 1, 2, and 3 described in the Examples later. In an SEM image, [Example 1] indicates a plate-shaped form which is entirely homogeneous and which has a crack, as shown in FIG. 1. In SEM images, [Example 2] and [Example 3] indicate a form wherein a plate-shaped homogeneous form is partly cracked. All of them are plate-shaped structures, most part of which shows continuity of a flat and homogeneous surface. The term “predominant” herein means a form by which an area of not less than 50% of a single visual field of an SEM image is occupied, and it is sufficient that this form is present either at the surface or in the inside of the drug coating layer as observed from a single balloon.

The surface of [Example 1] is shown in FIG. 1, is represented as 1(A) in Table 1 later, and has a substantially plate-shaped amorphous phase predominant therein.

The surface of [Example 2] is represented as 2(A) in Table 1 later, and has its an amorphous phase predominant therein.

The surface of [Example 3] is represented as 3(A) in Table 1 later, and has an amorphous phase predominant therein.

The drug coating layers (A) of 1(A), 2(A), and 3(A) are insusceptible to peeling in the process of delivery to a target tissue. An interaction between 3,5-dipentadecyloxybenzamidine (TRX-20), which is the amidine derivative, and cells may possibly enhance the tissue transferability of the drug.

[Second Form (B): SEM Photograph of the Drug Coating Layer in the Embodiment where Small Incomplete Crystals being about to be Crystals are Predominant]

The drug coating layer of form (B) wherein small incomplete crystals about to be crystals are predominant, although it has some amorphous phase, means a form wherein a plurality of small incomplete crystals about to be crystals are observed to occupy not less than 50% of the area of a single visual field of an SEM image. The surface is not flat but has ruggedness. This is exemplified, for example, by a surface or the like (shown in FIG. 2) of the drug coating layer [Example 4] obtained by coating a balloon surface with a first drug coating composition which is a coating solution 4 described in Example later. If such a form is present either at the surface or in the inside of the drug coating layer observed from a single balloon, the drug coating layer is (B) the embodiment where small incomplete crystals about to be crystals are predominant.

The surface of [Example 4] is shown in FIG. 2, is represented as 4(B) in Table 1 later, and has its small incomplete crystals (which are about to be crystals) predominant therein.

The drug coating layer (B) of the above-described 4(B) is insusceptible to peeling in the process of delivery to a target tissue. A crystalline form is observed in the drug coating layer, and, due to the characteristics of 3,5-dipentadecyloxybenzamidine (TRX-20) which is an amidine derivative, it is expected that the rate of target tissue transferability is enhanced.

### 4) Characteristics of the First Drug Coating Layer

4-1) The hydrophobic region corresponding to the alkyl chain length of the amidine derivative and a hydrophobic region of the water-insoluble drug show a hydrophobic interaction, and these hydrophobic regions have high affinity for the balloon surface. Therefore, the drug coating layer is insusceptible to peeling during the process of delivery to a target tissue, and can deliver a sufficient amount of drug to an affected area.

4-2) The positively charged moiety of the amidine derivative interacts with negatively charged cell surfaces, so that the drug coating layer is excellent in transferability of the water-insoluble drug to a target tissue.

### (2) Second Drug Coating Composition and Second Drug Coating Layer

1) The second drug coating composition is a composition in which the water-insoluble drug and the amidine derivative are present in the state of being dissolved in a solvent which contains glycerin.

### [Solvent]

Preferable solvents: tetrahydrofuran, ethanol and glycerin (also called glycerol or propane-1,2,3-triol) are used as solvents. Other solvents than the above-mentioned which can be added include acetone, methanol, dichloromethane, hexane, ethyl acetate and water.

### 2) Method of Preparing Second Drug Coating Composition

The amidine derivative is dissolved in tetrahydrofuran.

The water-insoluble drug is dissolved in ethanol, acetone, tetrahydrofuran, or a mixed solution thereof and glycerin.

Both of the solutions are prepared and then mixed. The glycerin may be added at the time of final mixing.

### 3) Second Drug Coating Layer

The second drug coating layer is formed by using the second drug coating composition on a surface of a medical device.

The relation between the solvent in the second drug coating composition and the resulting drug coating layer:

The resulting drug coating layer has a crystalline form which is predominant in the whole coating layer. Specifically, there can be obtained a third form (C) where small incomplete crystals in a network form are predominant, and a fourth form (D) where needle- or rod-shaped or spheroidal crystals are predominant. Where a crystal form of the third form or fourth form is present, this category includes a form wherein the surface is covered with an amorphous film and crystals are recognized in a portion underlying the film (the inside of the drug coating layer film).

It is considered that when glycerin is contained as solvent in the second drug coating composition, the rate of removal of the solvent is lowered, so that crystallization of the difficultly water-soluble drug is facilitated, and many crystals appear upon formation of the drug coating layer. In addition, when glycerin is present, crystallization is facilitated even in the presence of a large amount of tetrahydrofuran, which is highly volatile and is a good solvent for the amidine derivative and the water-insoluble drug. Further, when glycerin is contained and the content of ethanol or acetone is increased with respect to content of tetrahydrofuran, the form of crystals becomes definite, and a drug coating layer containing crystals densely is formed.

The drug coating layer [third form (C)] is a form which has small incomplete crystals in a network form in at least a portion thereof, and a preferable solvent ratio (mass %) is as follows.

| | | |
|---|---|---|
| THF | EtOH, Acetone, or | Glycerin |
| Not less than 75%, | EtOH/Acetone | Not less than 0.5%, |
| not more than 97% | mixed liquid | not more than 4% |
| | Not less than 0%, | |
| | not more than 25% | |

The drug coating layer [fourth form (D)] is a form which has needle- or rod-shaped or spheroidal crystals in at least a portion thereof, and a preferable solvent ratio (mass %) is as follows.

| | | |
|---|---|---|
| THF | EtOH, Acetone, or | Glycerin |
| Not less than 1%, | EtOH/Acetone | Not less than 0.5% |
| not more than 70% | mixed liquid | |
| | Not less than 29.5%, | |
| | not more than 98.5% | |

Third Form (C): SEM Photograph of Second Drug Coating Layer which has Small Incomplete Crystals in Network Form in at Least a Portion Thereof

The form of the surface or the inside of a drug coating layer of the third form (D) having small incomplete crystals in a network form in at least a portion thereof can be exemplified by surfaces or the like of drug coating layers [Example 5] (shown in FIG. 3) and [Example 6] obtained by coating a balloon surface with second drug coating compositions which are coating solutions 5 and 6 described in Examples later.

The embodiment where small incomplete crystals in a network form are predominant refers to a form wherein individual crystals are not present independently, it is difficult to determine the size of a single crystal, and crystals are joined in a network form, as in [Example 5]. In addition, this category also includes an embodiment where small incomplete crystals about to be crystals are recognized in the inside of the drug coating layer, although the surface of the layer is in a plate-shaped amorphous form, as in [Example 6]. Note that [Example 5] is an SEM image taken by an SEM.

The surface of [Example 5] is shown in FIG. 3, is represented as 5(C) in Table 1 later, and has small incomplete crystals in a network form present predominantly. The surface of [Example 6] is represented as 6(C) in Table 1 later, and has small incomplete crystals in a network form present predominantly.

[Fourth Form (D): SEM Photograph of Second Drug Coating Layer in the Embodiment where Crystalline Form is Predominant]

The form of the surface or the inside of a drug coating layer of the fourth form (D) wherein a crystalline form is predominant can be exemplified by surfaces or the like of drug coating layers [Example 7], [Example 8], and [Example 9] obtained by coating a balloon surface with second drug coating compositions which are coating solutions 7, 8, and 9 to be described in Examples later. As for the shape of crystals, various forms such as needle- or rod-shaped or spheroidal forms are observed either singly or in a mixed state.

D1) An SEM image of a drug coating layer [Example 7] where crystals (needle- or rod-shaped) are predominant is shown in FIG. 4.

The surface of [Example 7] is represented as 7(D1) in Table 1 later, and has crystals (needle- or rod-shaped) present predominantly therein.

D2) SEM images of a drug coating layer [Example 8] in the embodiment where crystals (spheroidal) are present predominantly or where the surface is covered with an amorphous film and spheroidal crystals are present in the inside are shown in FIGS. 5A and 5B. FIG. 5A is represented as 8(D2) in Table 1 later, and has crystals (spheroidal) present predominantly. The spheroidal crystals obtained in the present disclosure have surfaces which are not three-dimensional but are flat as if crushed down. FIG. 5B is represented as 8(D3) in Table 1 later, a surface thereof is covered with an amorphous film whereas spheroidal crystals are present in the inside thereof.

The surface of [Example 9] is represented as 9(D2) in Table 1 later, and has crystals (spheroidal) present predominantly therein.

4) The Second Drug Coating Layer is Characterized in that:

4-1) the second drug coating layer is insusceptible to peeling during the process of delivery to a target tissue, since the hydrophobic region of the alkyl chain length in the amidine derivative and the hydrophobic region of the water-insoluble drug show a hydrophobic interaction and the hydrophobic regions have high affinity for a balloon surface, so that a medical device capable of delivering a sufficient amount of drug to an affected area is provided; and

4-2) the second drug coating layer has crystals of the water-insoluble drug, and is excellent in transferability of drug to target tissue due to the interaction between the positively charged moiety of the amidine derivative and the negatively charged cell surfaces.

The medical device of the present disclosure has the drug coating layer formed either directly on a surface of a substrate or with a pretreatment layer (e.g., a primer layer) interposed therebetween. The drug coating layer contains the drug in an amount which is not particularly limited. The drug is contained in the drug coating layer in a density of 0.1 μg/mm² to 10 μg/mm², preferably 0.5 μg/mm² to 5 μg/mm², more preferably 0.5 μg/mm² to 3.5 μg/mm², and further preferably 1.0 μg/mm² to 3.0 μg/mm².

The shape of the substrate is not specifically restricted. The shape of the substrate, which is made of such a material as metal or resin, may be any of a film, a plate, a linear material, and a shaped section, or may be a granular or particulate shape.

The medical device to be used is not limited. The medical device may be any of implantable or insertable medical devices. The medical device is preferably an elongated medical device which is delivered in a radially reduced and non-expanded state within a body lumen or cavity such as a blood vessel and which is radially expanded in a circumferential direction in a local area such as a blood vessel, tissue or the like so that a drug is released from a drug coating layer. Therefore, the medical device delivered in a radially contracted state and applied to an affected area in a radially expanded state is a medical device having an expandable portion. The drug coating layer is provided at least at a portion of the surface of the expandable portion. Specifically, the drug is provided in a coating on at least the outer surface of the expandable portion.

The material of the expandable portion of the medical device preferably has a certain degree of flexibility and a certain degree of hardness such that the expandable portion can be expanded upon arrival at a blood vessel, tissue or the like to release the drug from the drug coating layer provided on the surface thereof. Specifically, the expandable portion is formed of metal or resin, and it is preferable that the surface of the expandable portion where the drug coating layer is provided is formed of a polymer. The polymer constituting the surface of the expandable portion is not specifically restricted, but is preferably a polyamide. Specifically, at least a portion of the surface of the expandable portion of the medical device to be coated with the drug is a polyamide. The polyamideis not particularly limited, so long as the polyamide is a polymer having an amide linkage. Examples of the polyamide include: homopolymers such as polytetramethylene adipamide (nylon 46), polycaprolactam (nylon 6), polyhexamethylene adipamide (nylon 66), polyhexamethylene sebacamide (nylon 610), polyhexamethylene dodecamide (nylon 612), polyundecanolactam (nylon 11), polydodecanolactam (nylon 12), etc.; and copolymers such as caprolactam/lauryllactam copolymer (nylon 6/12), caprolactam/aminoundecanoic acid copolymer (nylon 6/11), caprolactam/ω-aminononanoic acid copolymer (nylon 6/9), caprolactam/hexamethylenediammonium adipate copolymer (nylon 6/66), etc.; and aromatic polyamides such as a copolymer of adipic acid with metaxylenediamine, a copolymer of hexamethylenediamine with m,p-phthalic acid, etc. Further, polyamide elastomers which are block copolymers having a hard segment composed of nylon 6, nylon 66, nylon 11, nylon 12 or the like and a soft segment composed of polyalkylene glycol, polyether, or an aliphatic polyester or the like can also be used as a substrate of the medical device of the present disclosure. The polyamide may be used either singly or in combination of two or more of them.

Specific examples of the medical device having an expandable portion include expandable portions (stents) and elongated catheters having an expandable portion (balloon).

Preferably, the balloon in the present disclosure is formed on its surface when expanded with a drug coating layer of the present disclosure, is then wrapped (folded), is inserted into a blood vessel, a body cavity or the like, is delivered to a tissue or an affected area, and is radially expanded in the affected area to release the drug.

The present disclosure will be described below referring to Examples and Comparative Example, but the disclosure is not to be limited to these Examples. Note that in the following, all the Examples and Comparative Example will be described as Examples.

### 1. Fabrication of Drug Eluting Balloon

### Example 1

### (1) Preparation of Coating Solution 1

140 mg of 3,5-dipentadecyloxybenzamidine hydrochloride (TRX-20; Junsei Chemical Co., Ltd.; molecular weight: 609.41) was weighed, and was dissolved by adding 2 mL of tetrahydrofuran thereto, to prepare a 70 mg/mL TRX-20 solution. On the other hand, 168 mg of paclitaxel (Shanghai Zhongxi Sunve Pharmaceutical Co., Ltd.; molecular weight: 853.91) was weighed, and was dissolved by adding 2 mL of tetrahydrofuran (THF) and 1 mL of anhydrous ethanol (EtOH), to prepare a 56 mg/mL paclitaxel solution.

30 μL of the 70 mg/mL TRX-20 solution and 200 μL of the 56 mg/mL paclitaxel solution were admixed with each other, to obtain a coating solution 1 (having a mass ratio of TRX-20/PTX (WAN)=0.19/1 and a solvent ratio (by volume) of THF:EtOH=71:29).

### (2) Coating Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 1 by use of a pipette so that the amount of paclitaxel would be about 3 μg/mm², and the balloon was dried, to fabricate a drug eluting balloon having a drug coating layer [Example 1]. An SEM image of [Example 1] is shown in FIG. 1.

### Example 2

### (1) Preparation of Coating Solution 2

A 70 mg/mL TRX-20 solution and a 56 mg/mL paclitaxel solution were prepared in the same manner as in [Example 1].

11 μL of the 70 mg/mL TRX-20 solution and 200 μL of the 56 mg/mL paclitaxel solution were admixed with each other, to obtain a coating solution 2 (having a mass ratio of TRX-20/PTX (WAN)=0.07/1 and a solvent ratio (by volume) of THF:EtOH=68:32).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 2 by use of a pipette so that the amount of paclitaxel would be about 3 μg/mm², and the balloon was dried, to fabricate a drug eluting balloon having a drug coating layer [Example 2].

### Example 3

### (1) Preparation of Coating Solution 3

A 70 mg/mL TRX-20 solution was prepared in the same manner as in [Example 1].

112 mg of paclitaxel was weighed and was dissolved by adding 2 mL of tetrahydrofuran (THF) thereto, to prepare a 56 mg/mL paclitaxel solution.

11 μL of the 70 mg/mL TRX-20 solution and 200 μL of the 56 mg/mL paclitaxel solution were admixed with each other, to obtain a coating solution 3 (having a mass ratio of TRX-20/PTX (WAN)=0.07/1 and a solvent ratio (by volume) of THF=100).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 3 by use of a pipette so that the amount of paclitaxel would be about 3 μg/mm², and the balloon was dried, to fabricate a drug eluting balloon having a drug coating layer [Example 3].

### Example 4

### (1) Preparation of Coating Solution 4

A 70 mg/mL TRX-20 solution was prepared in the same manner as in [Example 1].

168 mg of paclitaxel was weighed and was dissolved by adding 1.5 mL of anhydrous ethanol (EtOH) and 1.5 mL of acetone thereto, to prepare a 56 mg/mL paclitaxel solution.

30 μL of the 70 mg/mL TRX-20 solution and 200 μL of the 56 mg/mL paclitaxel solution were admixed with each other, to obtain a coating solution 4 (having a mass ratio of TRX-20/PTX (WAN)=0.19/1 and a solvent ratio (by volume) of THF:EtOH:Acetone=13:43.5:43.5).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 4 by use of a pipette so that the amount of paclitaxel would be about 3 μg/mm², and the balloon was dried, to fabricate a drug eluting balloon having a drug coating layer [Example 4]. An SEM image of [Example 4] is shown in FIG. 2.

### Example 5

### (1) Preparation of Coating Solution 5

A 70 mg/mL TRX-20 solution was prepared in the same manner as in [Example 1].

1 g of glycerin (Glycerin, Kanto Chemical Co., Inc.; CAS No. 56-81-5) was weighed and was admixed with anhydrous ethanol so as to obtain a total amount of 2 g, thereby preparing a 50% glycerin solution.

112 mg of paclitaxel was weighed and was dissolved by adding 2 mL of tetrahydrofuran thereto, to prepare a 56 mg/mL paclitaxel solution.

60 μL of the 70 mg/mL TRX-20 solution, 17 μL of the 50% glycerin solution, and 200 μL of the 56 mg/mL paclitaxel solution were admixed with one another, to obtain a coating solution 5 (having a mass ratio of TRX-20/PTX (W/W)=0.38/1 and a solvent ratio (by volume) of THF:EtOH:Glycerin=94:3:3).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 5 by use of a pipette so that the amount of paclitaxel would be about 3 μg/mm², and the balloon was dried, to fabricate a drug eluting balloon having a drug coating layer [Example 5]. An SEM image of [Example 5] is shown in FIG. 3.

### Example 6

### (1) Preparation of Coating Solution 6

A 70 mg/mL TRX-20 solution was prepared in the same manner as in [Example 1].

A 50% glycerin solution was prepared in the same manner as in [Example 5].

112 mg of paclitaxel was weighed and was dissolved by adding 2 mL of tetrahydrofuran thereto, to prepare a 56 mg/mL paclitaxel solution.

11 μL of the 70 mg/mL TRX-20 solution, 6 μL of the 50% glycerin solution, and 200 μL of the 56 mg/mL paclitaxel solution were admixed with one another, to obtain a coating solution 6 (having a mass ratio of TRX-20/PTX (W/W)=0.07/1 and a solvent ratio (by volume) of THF:EtOH:Glycerin=97:1.5:1.5).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 6 by use of a pipette so that the amount of paclitaxel would be about 3 μg/mm², and the balloon was dried, to fabricate a drug eluting balloon having a drug coating layer [Example 6].

### Example 7

### (1) Preparation of Coating Solution 7

A 70 mg/mL TRX-20 solution was prepared in the same manner as in [Example 1].

A 50% glycerin solution was prepared in the same manner as in [Example 5].

168 mg of paclitaxel was weighed and was dissolved by adding 1.5 mL of anhydrous ethanol and 1.5 mL of acetone thereto, to prepare a 56 mg/mL paclitaxel solution.

30 μL of the 70 mg/mL TRX-20 solution, 15 μL of the 50% glycerin solution, and 200 μL of the 56 mg/mL paclitaxel solution were admixed with one another, to obtain a coating solution 7 (having a mass ratio of TRX-20/PTX (W/W)=0.19/1 and a solvent ratio (by volume) of THF:EtOH:Acetone:Glycerin=12:44:41:3).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 7 by use of a pipette so that the amount of paclitaxel would be about 3 μg/mm², and the balloon was dried, to fabricate a drug eluting balloon having a drug coating layer [Example 7]. An SEM image of [Example 7] is shown in FIG. 4.

### Example 8

### (1) Preparation of Coating Solution 8

A 70 mg/mL TRX-20 solution was prepared in the same manner as in [Example 1].

A 50% glycerin solution was prepared in the same manner as in [Example 5].

168 mg of paclitaxel was weighed and was dissolved by adding 1.5 mL of anhydrous ethanol and 1.5 mL of acetone thereto, to prepare a 56 mg/mL paclitaxel solution.

11 μL of the 70 mg/mL TRX-20 solution, 6 μL of the 50% glycerin solution, and 200 μL of the 56 mg/mL paclitaxel solution were admixed with one another, to obtain a coating solution 8 (having a mass ratio of TRX-20/PTX (W/W)=0.07/1 and a solvent ratio (by volume) of THF:EtOH:Acetone:Glycerin=5:47:46:1.5).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 8 by use of a pipette so that the amount of paclitaxel would be about 3 μg/mm², and the balloon was dried, to fabricate a drug eluting balloon having a drug coating layer [Example 8]. SEM images of [Example 8] are shown in FIGS. 5A and 5B.

### Example 9

### (1) Preparation of Coating Solution 9

A 70 mg/mL TRX-20 solution was prepared in the same manner as in [Example 1].

A 50% glycerin solution was prepared in the same manner as in [Example 5].

168 mg of paclitaxel was weighed and was dissolved by adding 1.5 mL of tetrahydrofuran and 1.5 mL of acetone, to prepare a 56 mg/mL paclitaxel solution.

35 μL of the 70 mg/mL TRX-20 solution, 24 μL of the 50% glycerin solution, and 600 μL of the 56 mg/mL paclitaxel solution, and 50 μL of acetone were admixed with one another, to obtain a coating solution 9 (having a mass ratio of TRX-20/PTX (WAN)=0.07/1 and a solvent ratio (by volume) of THF:EtOH:Acetone:Glycerin of 47:2:49:2).

### (2) Coating of Balloon with Drug

A balloon catheter (produced by Terumo Corporation; with a balloon portion (expandable portion) formed of nylon) with an expandable portion sized to be 3.0 mm in diameter and 20 mm in length when expanded was prepared. The balloon in an expanded state was coated with the coating solution 9 by use of a pipette so that the amount of paclitaxel would be about 3 μg/mm², and the balloon was dried, to fabricate a drug eluting balloon having a drug coating layer [Example 9].

### 2. Scanning Electron Microscopic (SEM) Observation of Drug Coating Layer on Drug Eluting Balloon

After the dried drug eluting balloons were cut to a suitable size, the cut piece was placed on a support base, and platinum vapor deposition was conducted from thereabove. Thereafter, the surface and the inside of each of the drug coating layers [Example 1] to [Example 9] were observed under a scanning electron microscope. Details of the drug coating layers [Example 1] to [Example 9] are set forth in Table 1, and SEM photographs are shown in FIGS. 1 to 5A and 5B. The [Example 10] as Comparative Example was a commercialized drug eluting balloon (IN.PACT) available from INVAtec Japan, in which an amorphous phase and crystals were mixedly present in the drug coating layer. SEM photographs of this are shown in FIGS. 6A and 6B. It is observed that this drug coating layer is substantially entirely amorphous, with needle-shaped crystal-like portions mixedly present.

(1) Examples concerning the first drug coating composition and the first drug coating layer are Examples 1 to 4.

[Example 1] to [Example 3], as represented by the SEM photograph in FIG. 1, have a plate-shaped amorphous phase present predominantly at the surface and in the inside of the whole coating layer, and has a flat plate-shaped structure. The Examples each had a coating layer wherein the surface is free of ruggedness (projections and recesses) and is homogeneous, and most portions are continuously connected to one another, though cracks are present in some portions. Further, the inside of the coating layer is also homogeneous and is free of crystals such as rod- or needle-shaped or spheroidal crystals.

[Example 4], as represented by the SEM photograph in FIG. 2, has a surface which is not homogeneous and not flat, but has ruggedness (projections and recesses) and incomplete crystals that are about to be crystals. The coating layer is free of gaps or cracks. The coating layer is not flat plate-shaped, and does not have a clearly profiled crystalline structure.

(2) Examples concerning the second drug coating composition and the second drug coating layer are Examples 5 to 9.

[Example 5] and [Example 6], as represented by the SEM photograph in FIG. 3, have small incomplete crystals in a network form. The individual crystals are not present independently, but are present in the state of being joined in a network form. Gaps (spaces) are present in the portion where the small incomplete crystals are joined in a network form.

[Example 7], as represented by the SEM photograph in FIG. 4, has rod- or needle-shaped crystals, which are so present that their longitudinal sides are lying along the balloon surface. The individual rod- or needle-shaped crystals are not present independently, and each crystal has a portion in contact with an adjacent crystal, so that spaces are not easily formed between the crystals.

[Example 8], as represented by the SEM photographs in FIGS. 5A and 5B, has spheroidal crystals, whose surfaces are not three-dimensional but are flat as if crushed down. Most of the crystals are rather in non-concentric spheroidal shapes than in concentric spheroidal shape. Although the individual spheroidal crystals are in close contact with one another, they are present independently. As shown in FIG. 5B, the surface may be covered with a plate-shaped amorphous film, with spheroidal crystals being present in the inside.

(3) The commercialized IN.PACT as Comparative Example is [Example 10].

[Example 10], as shown in FIGS. 6A and 6B, has amorphous phases and crystalline regions which are present mixedly. The coating layer is substantially entirely amorphous, with needle-shaped crystal-like regions observed to be mixedly present in a portion of the coating layer.

TABLE 1

| | | Mass ratio | |
|---|---|---|---|
| | | of PTX/TRX- | |
| | | 20(W/W) in | |
| Example | Component ratio of solvents | drug coating | Morphological |
| No. | in the coating composition | layer | form |
| Example 1, | THF: EtOH = 71:29 | 0.19 | FIG. 1, 1(A) |
| No. 1 | | | |
| Example 2, | THF: EtOH = 68:32 | 0.07 | 2(A) |
| No. 2 | | | |
| Example 3, | THF = 100 | 0.07 | 3(A) |
| No. 3 | | | |
| Example 4, | THF: | 0.19 | FIG. 2, 4(B) |
| No. 4 | EtOH: Acetone = | | |
| | 13:43.5:43.5 | | |
| Example 5, | THF: EtOH: Glycerin = | 0.38 | FIG. 3, 5(C) |
| No. 5 | 94:3:3 | | |
| Example 6, | THF: | 0.07 | 6(C) |
| No. 6 | EtOH: Glycerin = | | |
| | 97:1.5:1.5 | | |
| Example 7, | THF: | 0.19 | FIG. 4, 7(D1) |
| No. 7 | EtOH: Acetone: Glycerin = | | |
| | 12:44:41:3 | | |
| Example 8, | THF: | 0.07 | FIG. 5A, 8(D2), |
| No. 8 | EtOH: Acetone: Glycerin = | | FIG. 5B, 8(D3) |
| | 5:47:46:1.5 | | |
| Example 9, | THF: | 0.07 | 9(D2) |
| No. 9 | EtOH: Acetone: Glycerin = | | |
| | 47:2:49:2 | | |
| Example 10, | Eluting balloon on the | — | FIG. 6A, |
| No. 10 | market by | | FIG. 6B, |
| | INVAtec JAPAN | | 10(IN.PACT) |

### 3. Evaluation of Durability of Drug Coating Layer During Delivery Process by Use of Mimic Blood Vessel

Evaluations 1 and 2 as described below were conducted using the drug eluting balloons fabricated in Examples 4, 5, 7, and 8.

### (Evaluation 1)

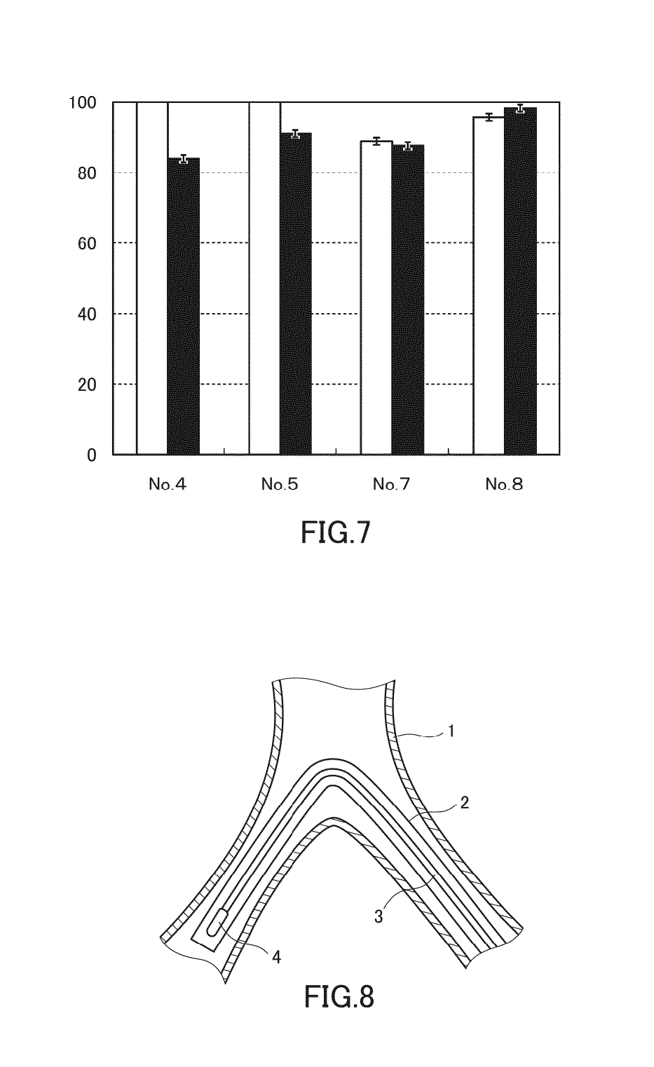

In order to evaluate how much of the drug is detached in the process of wrapping a balloon formed with a drug coating layer, the amount of paclitaxel remaining on the balloon surface after wrapping was measured. The remaining amount (μg) and the remaining rate (mass %) are set forth in Table 2. In addition, the remaining rate of PTX after wrapping is shown in FIG. 7.

### (Evaluation 2)

In order to evaluate how much of the drug coating layer with which the balloon surface is coated is detached during the process of delivery to a lesion affected area, a drug coating layer durability test was conducted using a mimic blood vessel.

A hollow mimic blood vessel 1 with a 90-degree angle shown in FIG. 8 was prepared, and a guiding catheter 2 (outside diameter: 5 Fr) was passed in the mimic blood vessel 1. The inside of the guiding catheter 2 was filled with phosphate-buffered saline (PBS) warmed to 37° C.

The drug eluting balloon after wrapping was subjected to a delivery operation. Specifically, a guide wire is inserted and passed in the guiding catheter 2 filled with the PBS (37° C.), then a balloon catheter 3 was inserted, and a delivering operation of delivering the balloon toward an outlet of the guiding catheter 2 was performed for one minute. The balloon 4 having been delivered was recovered, and the amount of paclitaxel remaining on the balloon portion was determined by liquid chromatography. The remaining amount (μg) and the remaining rate (mass %) are set forth in Table 2. In addition, the remaining rate of PTX after (wrapping+delivery) is shown in FIG. 7.

The durability of the drug coating layer during the wrapping operation and the delivery process was good, for any of the morphological forms.

In addition, even in the embodiment where glycerin was used as solvent, the durability of the drug coating layer during the wrapping operation and the delivery process was good.

TABLE 2

| | | Amount | Rate of | Amount | Rate of |
|---|---|---|---|---|---|
| | | of PTX | PTX | of PTX | PTX |
| | | remained | remained | remained | remained on |
| | | on a | on a | on a | a balloon |
| | | balloon | balloon | balloon after | after |
| | Drug | after | after | wrapping + | wrapping + |
| | coating | wrapping | wrapping | delivery | delivery |
| | layer | [μg] | [%] | [μg] | [mass %] |
| Example | Drug | 578.8 | 101 | 483.0 | 84 |
| 4, | coating | | | | |
| No. 4 | layer by | | | | |
| | coating | | | | |
| | solution 4 | | | | |
| Example | Drug | 620.1 | 105 | 539.0 | 91 |
| 5, | coating | | | | |
| No. 5 | layer by | | | | |
| | coating | | | | |
| | solution 5 | | | | |
| Example | Drug | 607.0 | 89 | 595.9 | 88 |
| 7, | coating | | | | |
| No. 7 | layer by | | | | |
| | coating | | | | |
| | solution 7 | | | | |
| Example | Drug | 651.0 | 96 | 665.0 | 98 |
| 8, | coating | | | | |
| No. 8 | layer by | | | | |
| | coating | | | | |
| | solution 8 | | | | |

The detailed description above describes a drug coating layer for a drug eluting medical device, a method of controlling the morphological form of a drug coating layer, a medical device, and a method of delivering a drug. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A drug coating layer formed on a substrate surface,
wherein the drug coating layer has at least one water-insoluble drug morphological form selected from the group consisting of the following morphological forms (1) to (4):
(1) a first morphological form in which a substantially plate-shaped amorphous phase is predominant at a surface and inside the wholedrug coating layer;
(2) a second morphological form in which small incomplete crystals are predominant;
(3) a third morphological form in which small incomplete crystals in a network form are present in at least a portion thereof; and
(4) a fourth morphological form in which needle- or rod-shaped or spheroidal crystals are present in at least a portion thereof.

2. The drug coating layer according to claim 1,
wherein the drug coating layer has at least one morphological form selected from the group consisting of the first morphological form and the second morphological form, namely,
(1) the first morphological form in which a substantially plate-shaped amorphous phase is predominant at a surface and inside the whole drug coating layer, and
(2) the second morphological form in which small incomplete crystals are predominant, and
the first morphological form and the second morphological form contain a same drug or different drugs, and the formation of the first morphological form and the second morphological form can be controlled by changing conditions for formation of the drug coating layer.

3. A method of controlling the morphological form of the drug coating layer according to claim 1,
wherein at the time of forming the drug coating layer by applying onto a substrate a drug coating composition which contains a water-insoluble drug and a glycerin-free solvent, rate of removal of the solvent is regulated.

4. The method of controlling the morphological form of the drug coating layer according to claim 3,
wherein the solvent is tetrahydrofuran, and ethanol.

5. The method of controlling the morphological form of the drug coating layer according to claim 3,
wherein the water-insoluble drug is rapamycin, paclitaxel, docetaxel, or everolimus.

6. The method of controlling the morphological form of the drug coating layer according to claim 3,
wherein the coating composition does not contain water.

7. A medical device which is provided on a surface thereof with the drug coating layer according to claim 1, is delivered in a radially contracted state when delivered in a living body, and is radially expanded locally so as to release the drug from the drug coating layer.

8. A method of delivering a drug, comprising:
delivering the medical device according to claim 7 into a lumen;
radially expanding an expandable portion possessed by the medical device; and
allowing the drug coating layer provided on the expandable portion to act on the lumen.

9. The method of controlling the morphological form of the drug coating layer according to claim 3,
wherein the morphological form of the drug coating layer is morphological form (1) and the solvent comprises 65 to 100 mass % tetrahydrofuran and 0 to 35 mass % of ethanol, acetone or a mixture of ethanol and acetone.

10. The method of controlling the morphological form of the drug coating layer according to claim 3,
wherein the morphological form of the drug coating layer is morphological form (2) and the solvent comprises 1 to 15 mass % tetrahydrofuran and 85 to 99 mass % of ethanol, acetone or a mixture of ethanol and acetone.

11. The method of controlling the morphological form of the drug coating layer according to claim 3,
wherein the morphological form of the drug coating layer is morphological form (3) and the solvent comprises 75 to 97 mass % tetrahydrofuran, 0 to 25 mass % of ethanol, acetone or a mixture of ethanol and acetone, and 0.5 to 4 mass % of glycerin.

12. The method of controlling the morphological form of the drug coating layer according to claim 3,
wherein the morphological form of the drug coating layer is morphological form (4) and the solvent comprises 1 to 70 mass % tetrahydrofuran, 29.5 to 98.5 mass % of ethanol, acetone or a mixture of ethanol and acetone, and not less than 0.5 mass % glycerin.
